(19) United States
(12) Patent Application Publication
(10) Pub. No.: US 2026/0045331 A1
(43) Pub. Date: Feb. 12, 2026

(54) CONTEXTUAL INFORMATION SYSTEM

(71) Applicant: Mamadou Fallilou Cissé, Bronx, NY (US)

(72) Inventor: Mamadou Fallilou Cissé, Bronx, NY (US)

(21) Appl. No.: 19/295,682

(22) Filed: Aug. 10, 2025

Related U.S. Application Data
(60) Provisional application No. 63/681,894, filed on Aug. 12, 2024.

(51) Int. Cl. G16H 10/60; A61B 90/96; G06F 3/14; G16H 40/20

(52) U.S. Cl. CPC G16H 10/60; A61B 90/96; G06F 3/14; G16H 40/20

(57) ABSTRACT

A contextual display device includes a housing including a mount extending from the housing that is configured for removable attachment to a mount bracket of an asset of a medical facility and a communication interface that is configured to communicate with a server that is configured to access patient data stored in an electronic health record system. The housing further includes a display that is configured to present contextual information and a processor in communication with the communication interface and the display. The processor is configured to cause the display to present a machine-readable code and obtain contextual information corresponding to the patient data from the server via the communication interface. The contextual display device is associated with the patient data by the server based on a scan of the machine-readable code by a scan device. The processor is further configured to cause the display to present the contextual information.

### CROSS REFERENCE TO RELATED APPLICATION

This application claims the benefit of U.S. Provisional Patent Application No. 63/681,894, entitled “J-Tag System for Enhancing Workflow Efficiency and Accuracy in Central Sterile Processing and Operating Room Environment,” filed on Aug. 12, 2024, which is incorporated by reference herein in its entirety.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND

This application relates to wireless display devices, and in particular, to a system and method for displaying dynamic, patient-specific information via battery-powered contextual display devices mounted to mobile assets, such as operating room case carts or other equipment.

In surgical environments, especially in high-volume hospitals, real-time communication of relevant patient information such as patient status, room assignments, and procedure timing is critical. Traditionally, paper-based signs or whiteboards having handwritten information or notes have been used to label case carts with the relevant patient information which may be used to inform perioperative staff of pending surgical cases.

The process of filling out the relevant patient information for each case cart is often completed manually by the staff and may not accurately reflect a current status of the patient information. As an example, updates to the patient information on the case cart may be prone to update delays due to the need for manual updating by a typically busy staff member. In addition, human error in transcribing the updated or original patient information to the case cart may result in inaccurate patient information on the case cart.

Differences in patient information between what is shown on the case cart and current patient information may result in a variety of delays in the care of a patient. As an example, a case cart with the wrong room assignment or an out-of-date room assignment may be sent to the wrong room, thereby potentially delaying surgery or another patient related activity. In another example, an error in the patient information on the case cart, e.g., a misspelled name, or other similar patient information, may need to be reconciled on the spot when a surgery is about to begin, before performing an operation which again may result in further delays.

### SUMMARY

In an embodiment, a contextual display device is disclosed. The contextual display device comprises a housing comprising a mount extending from the housing that is configured for removable attachment to a mount bracket of an asset of a medical facility and a communication interface that is configured to communicate with a server. The server is configured to access patient data stored in an electronic health record system. The housing further comprises a display that is configured to present contextual information and at least one processor in communication with the communication interface and the display. The at least one processor is configured to cause the display to present a machine-readable code and obtain contextual information corresponding to the patient data from the server via the communication interface. The contextual display device is associated with the patient data by the server based on a scan of the machine-readable code by a scan device. The at least one processor is further configured to cause the display to present the contextual information.

In an embodiment, the at least one processor is further configured to obtain updated contextual information corresponding to the patient data from the server via the communication interface and cause the display to present the updated contextual information.

In an embodiment, the contextual information corresponding to the patient data comprises contextual information about a status of the asset.

In an embodiment, the contextual information corresponding to the patient data comprises an instrumentation readiness status corresponding to a list of surgical instruments to be added to the asset.

In an embodiment, the contextual information corresponding to the patient data comprises location information corresponding to a location of the asset.

In an embodiment, the contextual information corresponding to the patient data comprises activity information corresponding to a use of the asset.

In an embodiment, the activity information comprises an operating room status corresponding to the use of the asset.

In an embodiment, a server is disclosed. The server comprises memory, a communication interface that is configured to interface with an electronic health record system, a contextual display device removably attached to an asset, and a scan device, and at least one processor that is configured to access patient data of a patient in the electronic health record system, obtain identifying information of the contextual display device from the scan device, the identifying information being generated based on a scan of a machine-readable code presented on a display of the contextual display device by the scan device, associate the contextual display device with the patient data based on the identifying information, obtain contextual information corresponding to the patient data from the electronic health record system via the communication interface, and transmit a command including the contextual information to the contextual display device, the command being configured to instruct the contextual display device to present the contextual information on the display.

In an embodiment, the at least one processor is further configured to obtain updated contextual information corresponding to the patient data from the electronic health record system via the communication interface and transmit a second command including the updated contextual information to the contextual display device, the second command being configured to instruct the contextual display device to present the updated contextual information on the display.

In an embodiment, the contextual information corresponding to the patient data comprises contextual information about a status of the asset.

In an embodiment, the contextual information corresponding to the patient data comprises an instrumentation readiness status corresponding to a list of surgical instruments to be added to the asset.

In an embodiment, the at least one processor is configured to obtain a location information corresponding to a location of the contextual display device via the communication interface, provide an update to the patient data corresponding to the location information and transmit a second command including updated contextual information corresponding the location information to the contextual display device the second command being configured to instruct the contextual display device to present the updated contextual information on the display including an indication of the location of the contextual display device.

In an embodiment, the contextual information corresponding to the patient data comprises activity information corresponding to a use of the asset.

In an embodiment, the activity information comprises an operating room status corresponding to the use of the asset.

In an embodiment, the obtaining the identifying information of the contextual display device from the scan device comprises providing an instruction to the scan device to scan the machine-readable code presented on the display of the contextual display device. The scan device is configured to present the instruction to a user of the scan device.

In an embodiment, a non-transitory computer-readable medium is disclosed. The non-transitory computer-readable medium stores instructions that, when executed by at least one processor, cause the at least one processor to access patient data of a patient in an electronic health record system and obtain identifying information of a contextual display device removably attached to an asset from a scan device. The identifying information is generated based on a scan of a machine-readable code presented on a display of the contextual display device by the scan device. The instructions further cause the at least one processor to associate the contextual display device with the patient data based on the identifying information, obtain contextual information corresponding to the patient data from the electronic health record system and transmit a command including the contextual information to the contextual display device, the command being configured to instruct the contextual display device to present the contextual information on the display.

In an embodiment, the instructions, when executed by the at least one processor, cause the at least one processor to obtain updated contextual information corresponding to the patient data from the electronic health record system and transmit a second command including the updated contextual information to the contextual display device, the second command being configured to instruct the contextual display device to present the updated contextual information on the display.

In an embodiment, the contextual information corresponding to the patient data comprises contextual information about a status of the asset.

In an embodiment, the contextual information about the status of the asset comprises at least one of an instrumentation readiness status corresponding to a list of surgical instruments to be added to the asset, location information corresponding to a location of the asset; and activity information corresponding to a use of the asset.

In an embodiment, obtaining the identifying information of the contextual display device from the scan device comprises providing an instruction to the scan device to scan the machine-readable code presented on the display of the contextual display device. The scan device is configured to present the instruction to a user of the scan device.

The foregoing summary is illustrative only and is not intended to be in any way limiting. These and other illustrative embodiments include, without limitation, apparatus, systems, methods and computer-readable storage media. In addition to the illustrative aspects, embodiments, and features described above, further aspects, embodiments, and features will become apparent by reference to the drawings and the following detailed description.

### BRIEF DESCRIPTION OF THE DRAWINGS

The invention is illustrated in the figures of the accompanying drawings which are meant to be exemplary and not limiting, in which like references are intended to refer to like or corresponding parts.

FIG. 1 is a block diagram of a contextual information system according to an embodiment.

FIG. 2 is a front view of a contextual display device of the contextual information system of FIG. 1 according to an embodiment.

FIG. 3 is a bottom-up view of the contextual display device of FIG. 2 according to an embodiment.

FIG. 4 is a side view of the contextual display device of FIG. 2 according to an embodiment.

FIG. 5 is a front perspective view of the contextual display device of FIG. 2 according to an embodiment.

FIG. 6 is a rear perspective view of the contextual display device of FIG. 2 according to an embodiment.

FIG. 7 is a perspective view of an asset of the contextual information system of FIG. 1 according to an embodiment.

FIG. 8 is a front perspective view of a mounting bracket of the contextual information system of FIG. 1 according to an embodiment.

FIG. 9 is a front view of the mounting bracket of FIG. 8 according to an embodiment.

FIG. 10 is a top-down view of the mounting bracket of FIG. 8 according to an embodiment.

FIG. 11 is a bottom-up view of the mounting bracket of FIG. 8 according to an embodiment.

FIG. 12 is a side view of the mounting bracket of FIG. 8 according to an embodiment.

FIG. 13 is a rear perspective view of the mounting bracket of FIG. 8 according to an embodiment.

FIG. 14 is a side assembly view of the contextual display device of FIG. 2 and the mounting bracket of FIG. 8 according to an embodiment.

FIG. 15 is a front view of another contextual display device of the contextual information system of FIG. 1 according to an embodiment.

FIG. 16 is a rear perspective view of the contextual display device of FIG. 15 according to an embodiment.

FIG. 17 is a perspective view of another mount bracket of the contextual information system of FIG. 1 according to an embodiment.

FIG. 18 is a perspective assembly view of the contextual display device of FIG. 15 and the mounting bracket of FIG. 17 prior to assembly according to an embodiment.

FIG. 19 is a perspective assembly view of the contextual display device of FIG. 15 and the mounting bracket of FIG. 17 after assembly according to an embodiment.

FIG. 20 is a perspective assembly view of the contextual display device of FIG. 15 and an asset of the contextual information system of FIG. 1 having the mounting bracket of FIG. 17 according to an embodiment.

FIG. 21 is a perspective assembly view of the contextual display device of FIG. 15 and the asset of FIG. 20 after assembly according to an embodiment.

FIG. 22 is a front perspective view of another contextual display device of the contextual information system of FIG. 1 according to an embodiment.

FIG. 23 is a front view of the contextual display device of FIG. 22 according to an embodiment.

FIG. 24 is a bottom-up view of the contextual display device of FIG. 22 according to an embodiment.

FIG. 25 is a side view of the contextual display device of FIG. 22 according to an embodiment.

FIG. 26 is a front view of another contextual display device of the contextual information system of FIG. 1 according to an embodiment.

FIG. 27 is a perspective assembly view of another mount bracket of the contextual information system of FIG. 1 attached to an asset according to an embodiment.

FIG. 28 is a perspective assembly view of the contextual display device of FIG. 22 and the mount bracket of FIG. 27 prior to attachment according to an embodiment.

FIG. 29 is a side assembly view of the contextual display device of FIG. 22 and the mount bracket of FIG. 27 prior to attachment according to an embodiment.

FIG. 30 is a side cross-sectional view of the contextual display device of FIG. 22 and the mount bracket of FIG. 27 prior to attachment according to an embodiment.

FIG. 31 is a perspective assembly view of the contextual display device of FIG. 22 and the mount bracket of FIG. 27 after attachment according to an embodiment.

FIG. 32 is a block diagram of a base station of the contextual information system of FIG. 1 according to an embodiment.

FIG. 33 is a side view of the base station of FIG. 32 according to an embodiment.

FIG. 34 is a perspective view of the base station of FIG. 32 according to an embodiment.

FIG. 35 is a perspective view of the base station of FIG. 32 with multiple contextual display devices of FIG. 22 attached according to an embodiment.

FIG. 36 is a side view of the base station of FIG. 32 with multiple contextual display devices of FIG. 22 attached according to an embodiment.

FIGS. 37-40 are front views of the contextual display device of FIG. 22 presenting contextual information about battery states of the contextual display device according to an embodiment.

FIGS. 41 and 42 are front views of the contextual display device of FIG. 22 presenting contextual information about an instrumentation status of an attached asset according to an embodiment.

FIGS. 43 and 44 are front views of the contextual display device of FIG. 22 presenting contextual information about a location or movement of an attached asset according to an embodiment.

FIGS. 45-47 are front views of the contextual display device of FIG. 22 presenting contextual information about a usage of the asset in the context of a surgical procedure according to an embodiment.

FIG. 48 is a front view of the contextual display device of FIG. 22 presenting contextual information about a post procedure state of the contextual display device according to an embodiment.

FIG. 49 is a signal diagram of communications between a contextual display device, a scan device, a server and an EHR system of the contextual information system of FIG. 1 according to an embodiment.

### DETAILED DESCRIPTION OF THE INVENTION

Subject matter will now be described more fully hereinafter with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. It is to be understood that other embodiments may be utilized and structural changes may be made without departing from the scope of the illustrative embodiments. Likewise, a reasonably broad scope for claimed or covered subject matter is intended. Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of exemplary embodiments in whole or in part. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

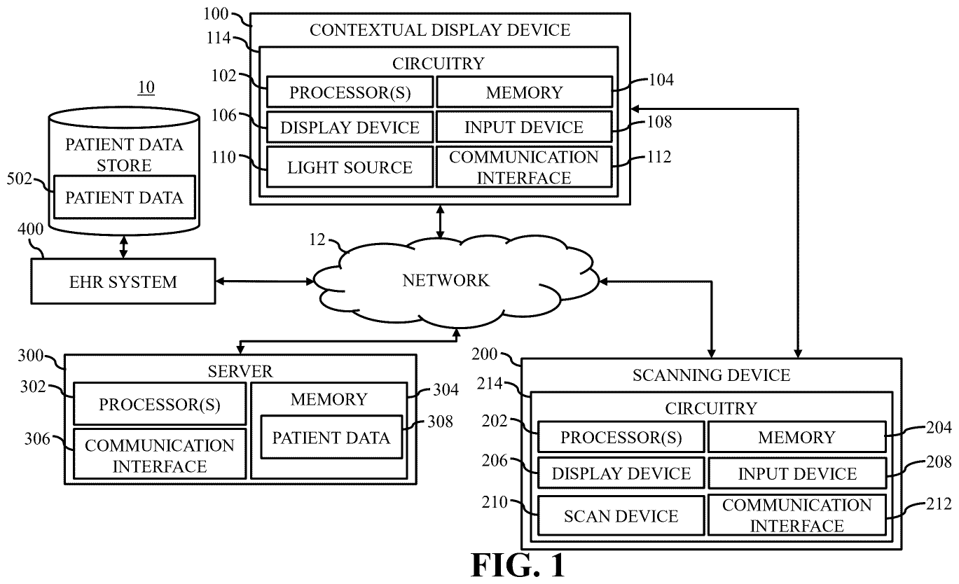

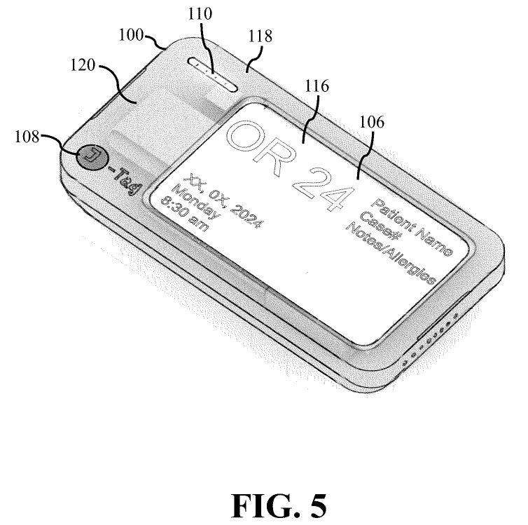

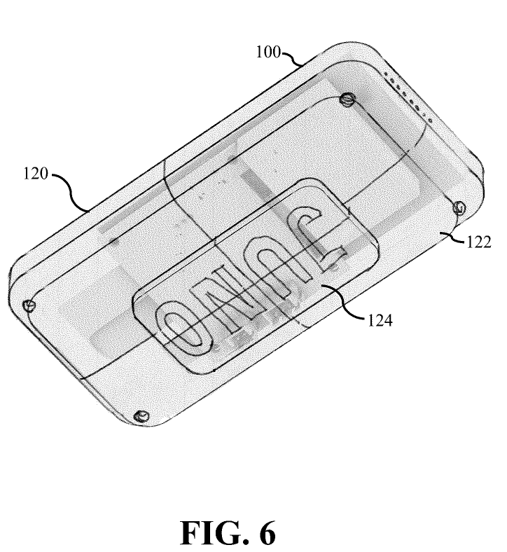

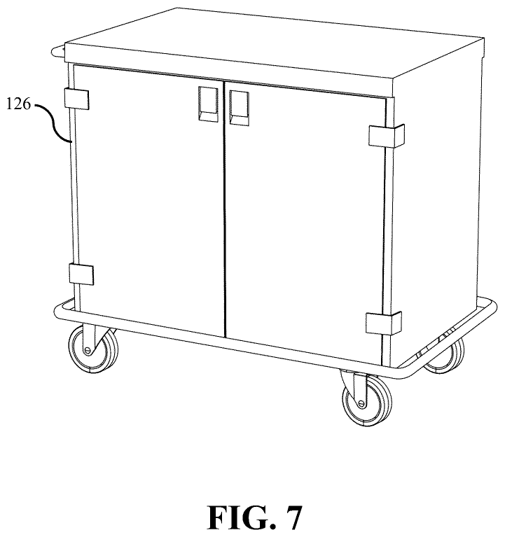

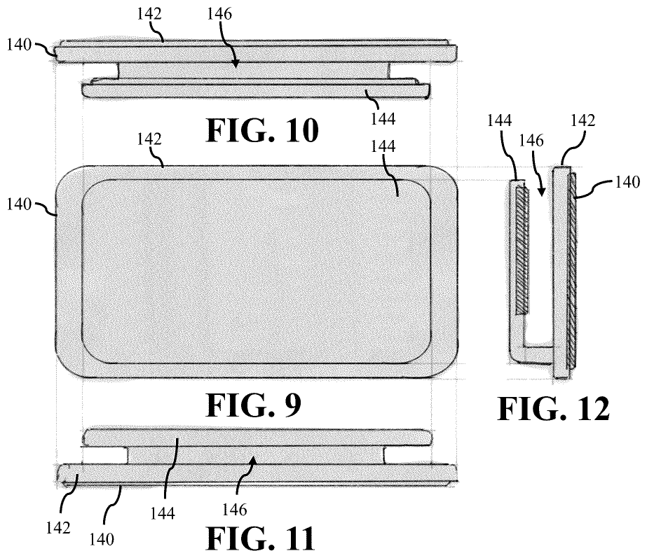

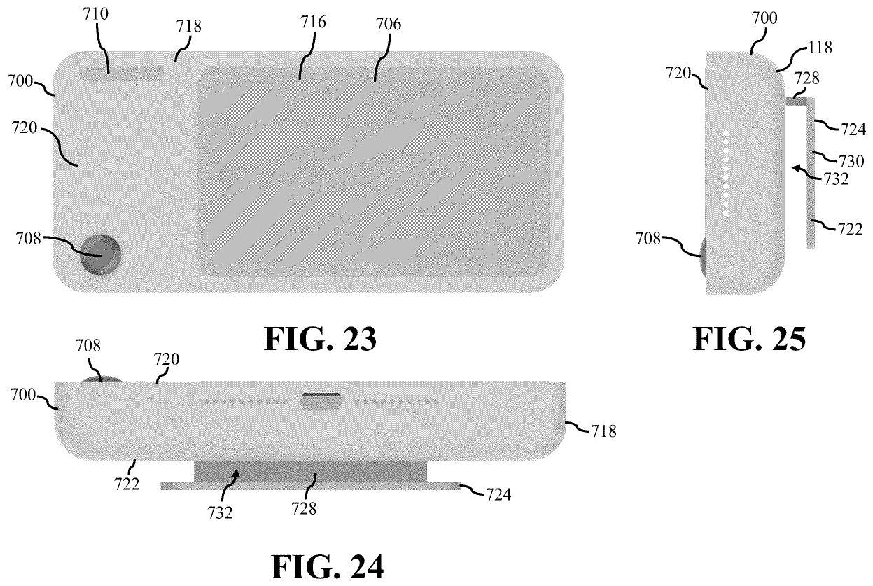

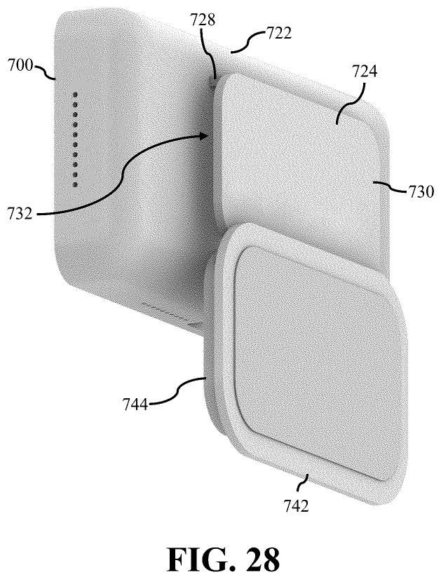

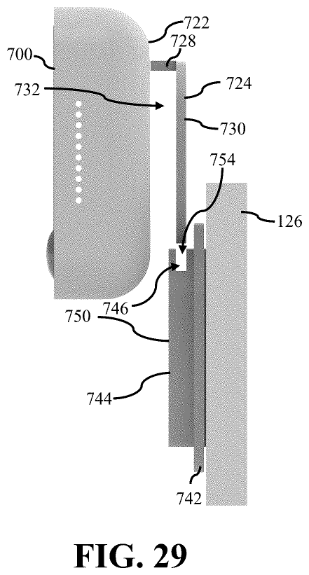

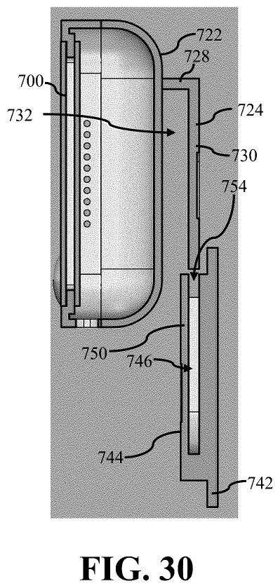

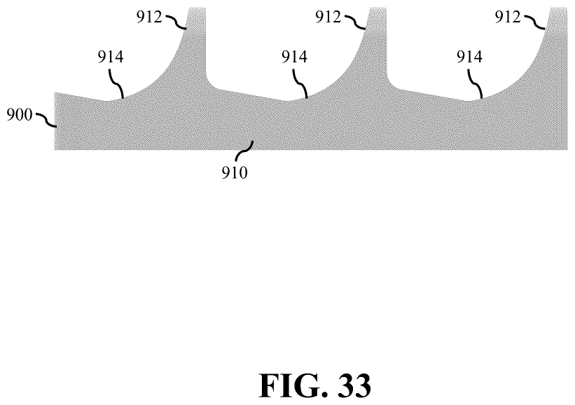

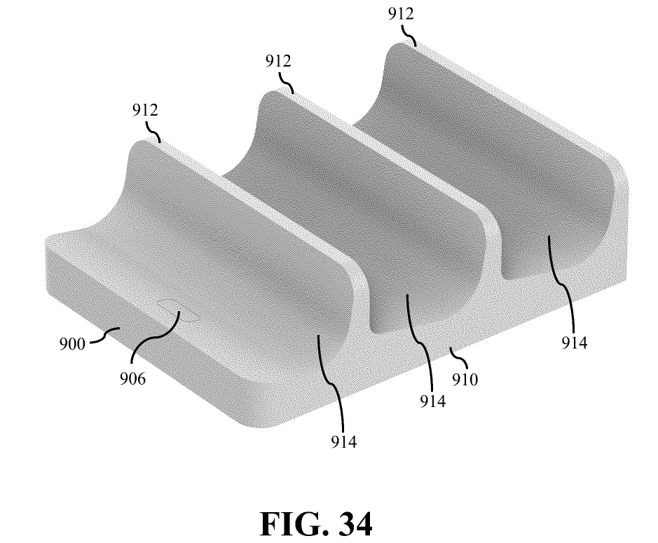

With reference to FIGS. 1-49, a contextual information system 10 is disclosed.

With reference to FIG. 1, contextual information system 10 comprises a network

12, one or more contextual display device(s) 100, one or more scanning devices(s) 200, a server 300, an electronic health record (EHR) system 400 and a patient data store 500.

Network 12 is configured to connect contextual display device(s) 100, scanning devices(s) 200, server 300 and EHR system 400 together and comprises one or more wired, wireless or combined wired/wireless networks and corresponding hardware such as hubs, switches, access points, network interfaces or other hardware commonly found in a network. Example wired and wireless networks that may be utilized include the Internet, a wide area network (WAN), a local area network (LAN), satellite, telephone, cable, a fiber-optic, cellular, ethernet, Wi-Fi, WiMAX, Bluetooth®, any other network or connection or any combination thereof.

With reference to FIG. 1, a contextual display device 100 comprises circuitry or other electronic components including, for example, one or more of processor(s) 102, memory 104, a display 106, an input device 108, a light source 110 and a communication interface 112, all of which may be collectively referred to herein as circuitry 114.

Processor 102 comprises, e.g., a processor, a microprocessor, a microcontroller, an application-specific integrated circuit (ASIC), a field-programmable gate array (FPGA), a graphics processing unit (GPU), a printed circuit board (PCB) or any other type of processing circuitry, as well as portions or combinations of such circuitry elements. In some embodiments, circuitry specifically configured to perform one or more functions of contextual display device 100 may also or alternatively be included, for example, instead of or in addition to a processor 102.

Memory 104 comprises, e.g., random access memory (RAM), read-only memory (ROM), flash memory or other types of memory, in any combination. The memory and other memories disclosed herein should be viewed as illustrative examples of what are more generally referred to as “processor-readable storage media” storing executable program code, registers, or other data that are configured for use by contextual display device 100 in performing one or more functions of contextual display device 100. In some embodiments, memory 104 may be configured to store data obtained from server 300 only to the extent that the data is utilized to update display device 106 and optical substrate 116 after which the data may be purged aside from that presented on optical substrate 116.

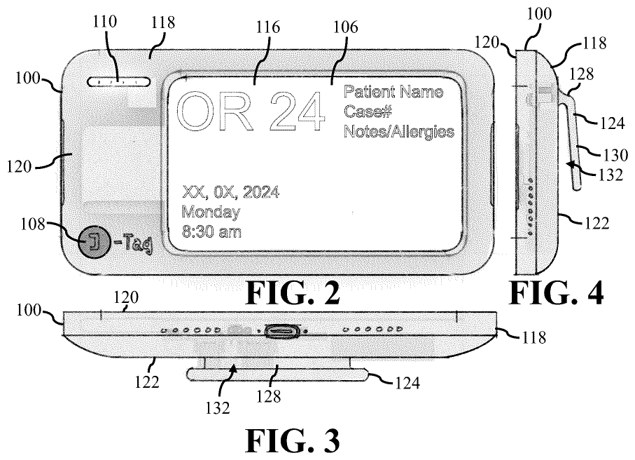

Display 106 comprises screen, panel or another optical substrate 116 (FIG. 2) that may be configured to change state in some embodiments. For example, processor 102 may be configured to control display 106 to effect a change in state or other optical properties of optical substrate 116, e.g., by adjusting the color, transparency or other properties of some or all of optical substrate 116. Display 106 comprises a driver circuit that is configured to apply voltage, current or an electric field to optical substrate 116, e.g., electrochromic, Polymer Dispersed Liquid Crystal (PDLC), Suspended Particle Device (SPD), Electrophoretic Display (EPD), Electrowetting Display (EWD) or another E-paper, smart glass or changeable optical substrate technology. Display 106 is configured to modulate various properties of optical substrate 116 including, e.g., the tint, hue, opacity or other properties, based on environmental conditions, user preferences, system states or commands other parameters. Memory 104 may, in some embodiments, store predefined display profiles or transition sequences for use by processor 102 to control display 106.

In an embodiment, optical substrate 116 comprises an E-paper panel such as, e.g., an E-ink panel. In other embodiments, other types of E-paper like panels may alternatively be utilized including, e.g., an Electrophoretic Display, an Electrowetting Display (EWD), an Electrochromic Display, a Cholesteric Liquid Crystal (ChLCD), Interferometric Modulator Display (IMOD) or any other E-paper like display. For example, the selected E-paper panel is configured to maintain the display state without further battery or power usage or with small amounts of battery or power usage after a change in display state.

In other embodiments, optical substrate 116 may alternatively comprise a powered display such as, e.g., an Organic Light-Emitting Diode (OLED) display, an Active Matrix OLED (AMOLED) display, a Liquid Crystal Display (LCD) such as, e.g., a Twisted Nematic (TN) LCD, In-Plane Switching (IPS) LCD or Thin-Film Transistor (TFT) LCD, a Mini Light Emitting Diode (Mini LED) or microLED display, a Transflective LCD, a LED Matrix or Segment display or any other powered or partially-powered display.

Input device 108 comprises, e.g., a touch sensitive screen, pad, button or other element of contextual display device 100 that may be actuated by a user to change a state of contextual display device 100. As an example, input device 100 may be actuated by a user to control display 106 to cycle through various display states for optical substrate 116. An example input device 108 in the form of a button is shown in FIG. 2. In some embodiments, optical substrate 116 may also or alternatively comprise a touch-sensitive input device 108.

Light Source 110 comprises an LED or another technology that may change color to indicate a current status of contextual display device 100. As an example, light source 110 may be configured to transition between a variety of colors states including, e.g., off, while, green, yellow, red or any other color to present a user of contextual display device 100 with information on a status of contextual display device 100, e.g., battery level, readiness for use, current status of the item for which contextual display device 100 is providing contextual information, or any other information.

Communication interface 112 is configured to actively or passively communicate and connect with external devices such as scanning device 200, server 300 or even EHR system 400 via one or more short-range or long-range communication protocols. As an example, communication interface 112 may comprise a transceiver or other technology that is configured to support local connectivity such as, e.g., Bluetooth, Blue Tooth Low Energy (BLE), Near Field Communication (NFC), Zigbee, Z-Wave, Ultra-Wideband (UWB), Infrared (IR), Wi-Fi Direct, or other proprietary low-power wireless protocols and technology. As another example, communication interface 112 may comprise a transceiver or other technology that is configured to support mid-range and long-range connectivity including, e.g., Wi-Fi, cellular network, satellite such as Iridium, Starlink and the like, Low-Power Wide-Area Networks (LPWAN), Narrowband IoT (NB-IoT), ultra-narrowband IoT communications such as Sigfox, or other communication technologies. Communication interface 112 may comprise one or more of the above-mentioned technologies alone or in any other combination.

Communication interface 112 may be configured to receive data and information from scanning device 200, server 300 or EHR 400 for presentation by display 106 and may be configured to periodically broadcast identifying information, receive control commands or location beacons, and transmit telemetry data indicative of contextual display device 100's status and location to connected devices such as scanning device 200, server 300 and EHR400. Communication interface 112 may be configured to support geofencing operations by detecting entry into or exit from predefined virtual boundaries, e.g., hospital rooms, floors, hallways, or other locations, in conjunction with another device such as, e.g., scanning device 200, server 300 or other devices, e.g., beacons or other fixed stations within a hospital.

Communication interface 112 may be configured to connect securely with server 300, e.g., via Wi-Fi or another technology, and receive a payload from server 300. The payload may comprise, for example, a JSON payload or any other type or protocol of payload including contextual information to be presented on optical substrate 116.

With reference again to FIG. 1, scanning device 200 comprises one or more processor(s) 202, memory 204, a display 206, an input device 208, a scan device 210 and a communication interface 212. In some embodiments, scanning device 200 may comprise a specially configured scanning device that is configured to scan a machine-readable code such as, e.g., a hospital barcode reader, or may comprise a general-purpose scanning device 200 such as, e.g., a mobile phone, tablet, camera or other similar device.

As an example, scanning device 200 may comprise a scanning device connected to a desktop computer or mobile computer cart/nurses station, a stand-alone wireless scanning device 200 having wireless communication capabilities and connected to server 300 or EHR 400, a mobile device or any other device that may be utilized to scan or update a status of contextual display device 100 to server 300 or EHR system 400.

Processor(s) 202 may comprise, e.g., a processor, a microprocessor, a microcontroller, an application-specific integrated circuit (ASIC), a field-programmable gate array (FPGA), a graphics processing unit (GPU), a printed circuit board (PCB) or any other type of processing circuitry, as well as portions or combinations of such circuitry elements.

Memory 204 may comprise, e.g., random access memory (RAM), read-only memory (ROM), flash memory or other types of memory, in any combination. The memory and other memories disclosed herein should be viewed as illustrative examples of what are more generally referred to as “processor-readable storage media” that may store executable program code of one or more software programs. Memory 204 may store one or more programs or applications that may be utilized by a user to process information received from a scan of contextual display device 100 and provide that information to server 300 or EHR system 400.

Display 206 comprises, e.g., a screen, a monitor, a television, phone screen, smart glass, E-paper or any other technology that is configured to present data or images to a user of scanning device 200.

Input device 208 comprises, e.g., a keyboard, mouse, touch screen, or any other physical interface that is configured to receive a user input from a user.

Scan device 210 is configured to capture a machine-readable code such as, e.g., a barcode, QR code, data matrix or other image from contextual display device 100. As an example, where scanning device 200 is a specialized handheld scanner or specialized or ruggedized mobile computer or tablet, scan device 210 may comprise a 2D image scanner for scanning identifying information from contextual display device 100. In another example, where scanning device 200 comprises a smartphone or other mobile device, scan device 210 may comprise an integrated camera running a scanning Software Development Kit (SDK) such as, e.g., Scandit or Zebra Data Wedge.

Communication interface 212 is configured to wirelessly communicate with one or more of contextual display device 100, server 300 and EHR system 400 either directly on a device-to-device or peer-to-peer basis or via network 12 using one or more of the communication protocols and technologies described above for communication interface 112.

With reference again to FIG. 1, server 300 comprises one or more processor(s) 302, memory 304, and a communication interface 306.

Processor(s) 302 may comprise, e.g., a processor, a microprocessor, a microcontroller, an application-specific integrated circuit (ASIC), a field-programmable gate array (FPGA), a graphics processing unit (GPU), a printed circuit board (PCB) or any other type of processing circuitry, as well as portions or combinations of such circuitry elements.

Memory 304 may comprise, e.g., random access memory (RAM), read-only memory (ROM), flash memory or other types of memory, in any combination. The memory and other memories disclosed herein should be viewed as illustrative examples of what are more generally referred to as “processor-readable storage media” that may store executable program code of one or more software programs.

Communication interface 306 is configured to wirelessly communicate with one or more of contextual display device 100, scanning device 200 and EHR system 400 via network 12 using one or more communication protocols and technologies such as those described above for communication interface 112.

With reference again to FIG. 1, EHR system 400 comprises a computerized platform that securely collects, stores, manages, and transmits patient data 502 such as patient health information, patient medical records, patient preference cards, or other patient information in a digital format. EHR system 400 integrates with and manages access to patient data store 500 in which the patient data 502 is stored. EHR system 400 is configured to integrate patient data 502 from various healthcare providers and departments, such as vital signs, lab results, medical imaging, prescriptions, treatment history, surgical procedures, scheduled appointments and surgeries, or any other patient data into a unified, real-time accessible record of patient data 502 stored in patient data store 500. EHR system 400 integrates with contextual display device 100, scanning device 200 and server 300 to present contextual information related to the patient data or hospital on display 106 of contextual display device 100.

As an example, in a case where contextual display device 100 is configured for use with a surgical case cart, the contextual information may include one or more of 1) patient name, 2) case number, 3) notes/allergies, 4) operating room assignment, 5) instrumentation progress, 6) transport status, 7) location or location related information, 8) surgery status, 9) post surgery status or cleaning status, or any other patient or hospital related information that is relevant to provide a context for surgical case cart to a nurse, doctor or other hospital staff member.

In some embodiments, contextual display device 100 may be configured to control the manner of presentation patient data 502 as contextual information or which patient data 502 is displayed in compliance with HIPPA rules and regulations. For example, where HIPPA rules and regulations require a staff member to be present during the presentation of certain patient data 502 as contextual information, such as a patient name or medical notes about a patient's status, contextual display device 100 may require an authorization to display that information, e.g., by a scan of a machine-readable code such as, e.g., barcode, QR code or other code, followed by a backend authentication with EHR system 400 for that staff member. Upon receipt by contextual display device 100 of a valid authorization, e.g., as received from server 300 or directly from EHR system 400, contextual display device 100 may present the certain patient data 502 as contextual information to the user.

In some embodiments, after a predetermined amount of time, e.g., a timeout period, the contextual information presented by contextual display device 100 may revert or otherwise change to contextual information that is allowed to be viewed under HIPPA rules or regulations in the absence of a staff member. For example, if optical substrate 116 is an E-paper technology, the contextual information would normally stay presented. In this embodiment, contextual display device 100 may actively change the contextual information presented on optical substrate 116 after the timeout period has elapsed without further user input or scanning of contextual display device 100.

With reference to FIGS. 1-6, contextual display device 100 comprises a housing 118 including circuitry 114. Contextual display device 100 and housing 118 may also comprise other common components such as, e.g., a charging port, ventilation holes, wireless charging technology or other similar components.

Housing 118 comprises a front housing 120 and a rear housing 122. Front housing 120 and rear housing 122 may comprise separate components that are joined together or alternatively may comprise two portions of a single component.

Front housing 120 comprises input device 108, optical substrate 116 of display 106 and light source 110. Rear housing 122 comprises a mount 124 that is configured for attachment to an asset 126 (FIG. 7) in need of contextual information such as, e.g., a surgical case cart as shown. Asset 126 may comprise any other object for which the presentation of contextual information may be useful. In the context of a hospital, example assets 126 may include hospital beds, stretchers, wheelchairs, transport chairs, IV poles, pumps, dispensers, monitors, portable imaging devices such as ultrasound, x-ray or other devices, telemetry units and patient monitors, surgical instruments, trays and toolkits, point-of-care testing kits, specimen containers and transport carriers, medication carts, anesthesia carts, crash carts, linen and supply bins, mobile workstations, portable air filters or HVAC units (for isolation rooms as an example), sanitization robots, UV cleaning tools, defibrillators (AEDs), patient room signage, isolation room signage, surgical or operating room signage or any other portion of a hospital that may be in need of contextual information that may be updated or changed for a variety of reasons. Some example reasons include new patient, staff, room, or other assignments, the association of an asset 126 with a particular individual, staff member, or location, or for any other reason. Assets 126 may comprise unique machine-readable codes that may be scanned, e.g., by scanning device 200, to identify asset 126 and associate asset 126 with other components of contextual information system 10 such as, e.g., a particular contextual display device 100, patient data 502 or any other component.

Mount 124 comprises a flange 128 extending outward from rear housing 122 of housing 118 and an arm 130 extending from flange 128. Rear housing 122, flange 128 and arm 130 together define a slot 132. Other types of mounts 124 may alternatively be utilized for attachment of contextual display device 100 to an asset 126 including, e.g., hook and loop fasteners, a snap-fit arrangement, a magnetic attachment, brackets, suction cups, rubber straps or bands, slide-in rails or tracks or any other attachment mechanism.

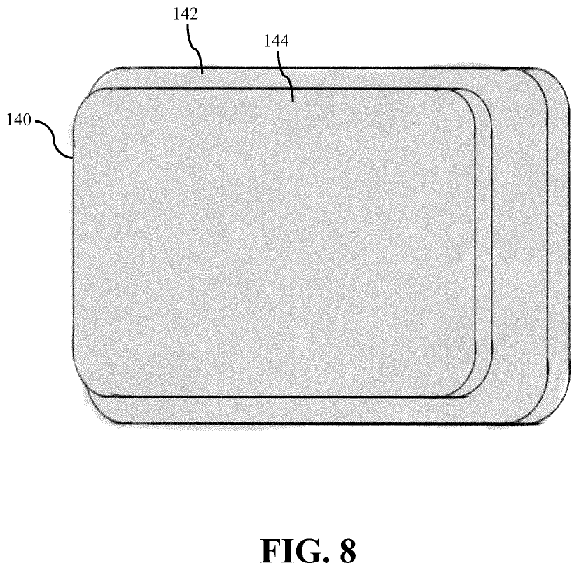

With reference to FIGS. 8-14, an example mount bracket 140 that is configured to receive mount 124 for removable attachment of contextual display device 100 to asset 126 will be described.

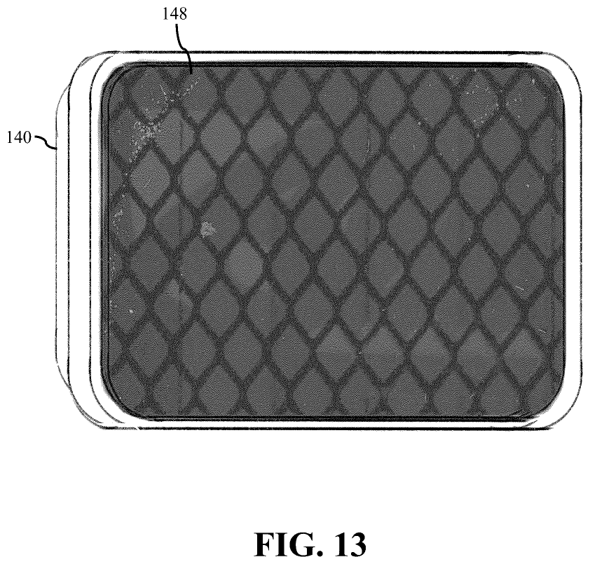

Mount bracket 140 comprises a base plate 142 and an arm 144 that together define a slot 146 therebetween for receiving a portion of arm 130 of mount 124 as shown in FIG. 13. Arm 144 is also configured for positioning within slot 132 of mount 124. Mount bracket 140 may be secured to asset 126 using an attachment mechanism 148, for example, an adhesive, hook and loop fasteners, a snap-fit arrangement, a magnetic attachment, brackets, suction cups, slide-in rails or tracks or any other attachment mechanism. In the embodiment shown in FIG. 12, attachment mechanism 148 comprises an adhesive.

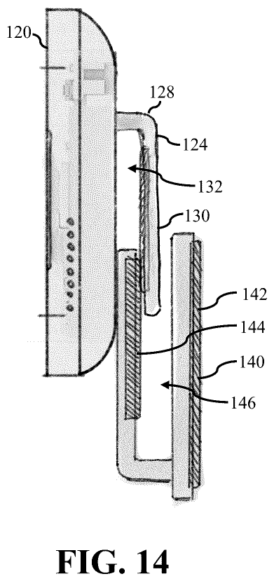

As shown in FIG. 14, housing 118 is secured to mount bracket 140 by the insertion of arm 130 of mount 124 into slot 146 of mount bracket 140, and the insertion of arm 144 of mount bracket 140 into slot 132 of mount 124. In this manner, contextual display device 100 may be removably attached to asset 126 and provide contextual information to a user of asset 126 such as hospital staff or any other user in need of contextual information about asset 126.

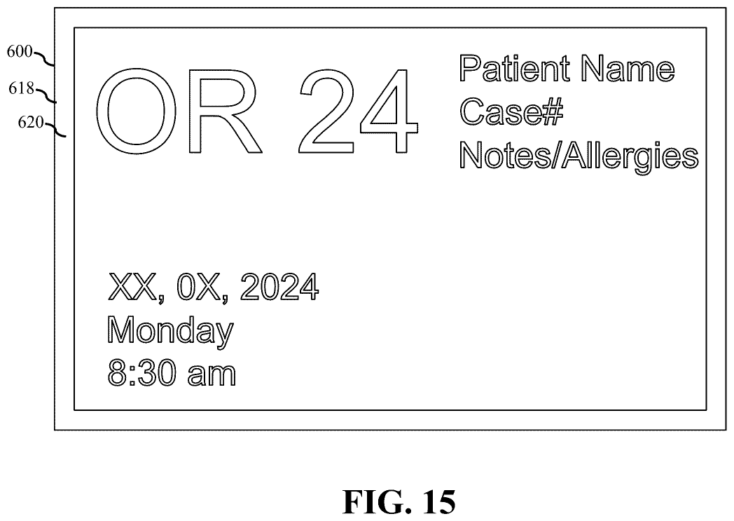

With reference to FIGS. 15-26, an example contextual display device 600 according to another embodiment is described. Contextual display device 600 comprises the same or similar functionality and components to contextual display device 100 except as described below where like components have similar numbering.

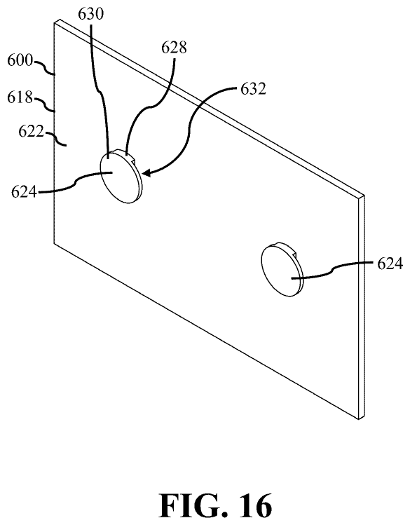

With reference to FIGS. 15 and 16, for example, contextual display device 600 comprises a housing 618 including a front housing 620 and a rear housing 622. Rear housing 622 includes a pair of mounts 624, for example, dovetail mounts, each comprising a rail 628 extending outward from rear housing 622 and a face plate 630 disposed on rail 628 to define one or more slots 632 between rail 628, face plate 630 and rear housing 622. While two mounts 624 are shown in FIGS. 15 and 16, any other number of mounts may alternatively be utilized including one mount 624, three mounts 624, four mounts 624 or any other number of mounts 624.

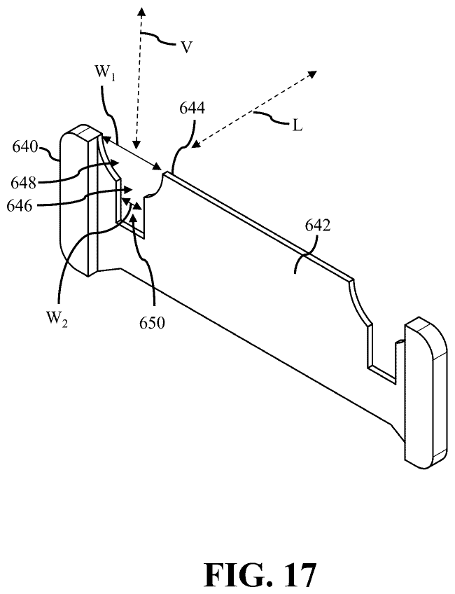

With reference to FIG. 17, a mount bracket 640 includes body 642 including a slot 646 that is configured to receive rail 628 of mount 264. Any number of slots 646 may be included, e.g., depending on the number of mounts 624. Body 642 and slot 646 are dimensioned to receive an insertion of rail 628 of mount 624 in a first direction, e.g., a vertical direction V, while inhibiting movement of mount 624 from slot 626, in a lateral direction L, once inserted due to engagement of face plate 630 against body 642. As shown in FIG. 17, for example, slot 646 may comprise a first opening 648 extending from an edge 644 of body 642 having a first width W₁ and a second opening 650 extending into body 642 from first opening 648 and having a second width W₂ that is smaller than first width W₁. In some embodiments, a width of first opening 648 may taper or otherwise progressively narrow from width Wi at edge 644 of body 642 to width W₂ at second opening 650. In some embodiments, second opening 650 may comprise parallel walls of uniform width that are sized to match and receive rail 628. Mount bracket 640 may be attached to asset 126 in any manner such as those described above for mount bracket 140.

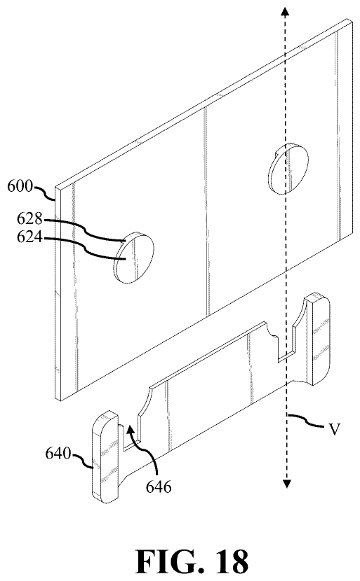

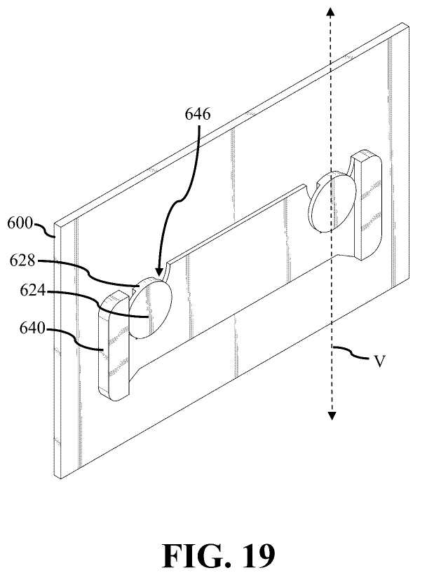

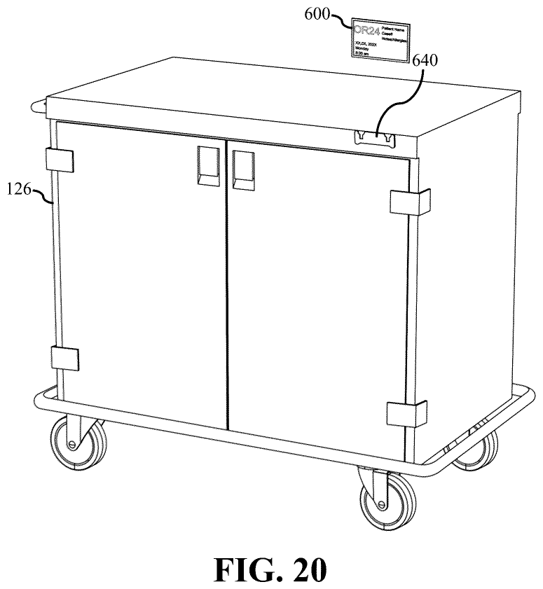

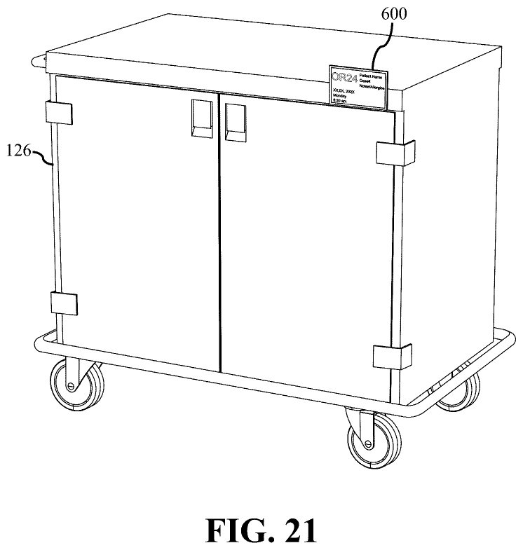

With reference to FIGS. 18-21, an attachment of contextual display device 600 to mount bracket 640 is illustrated. As shown in FIGS. 18 and 20, contextual display device 600 is positioned over mount bracket 640, e.g., in vertical direction V, such that mounts 624 of contextual display device 600 are aligned with slots 646 of mount bracket 640. Contextual display device 600 is then translated in vertical direction V to insert rails 628 of mounts 624 into slots 646 of mount bracket 640 to attach contextual display device 600 to asset 126, e.g., as shown in FIGS. 19 and 21.

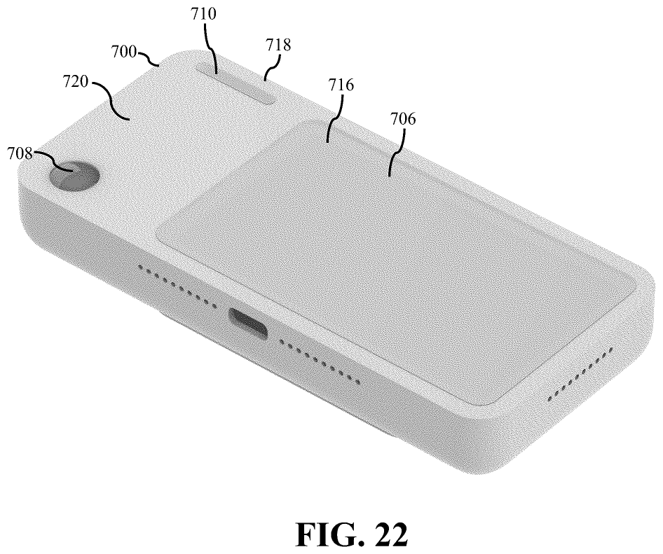

With reference to FIGS. 22-24, a contextual display device 700 is described. Contextual display device 700 comprises similar features to contextual display device 100 where like reference numbers refer to similar components and functionality except as described below.

Contextual display device 700 comprises a housing 718 including the same or similar circuitry 114 (FIG. 1) to that found in contextual display device 100. Contextual display device 700 and housing 718 may also comprise other common components such as, e.g., a charging port, ventilation holes, wireless charging technology or other similar components.

Housing 718 comprises a front housing 720 and a rear housing 722. Front housing 720 and rear housing 722 may comprise separate components that are joined together or alternatively may comprise two portions of a single component.

Front housing 720 comprises an input device 708, an optical substrate 716 of a display 706 and a light source 710. Rear housing 722 comprises a mount 724 that is configured for attachment to an asset 126 (FIG. 7) in need of contextual information such as, e.g., a surgical case cart as shown.

Mount 724 comprises a flange 728 extending outward from rear housing 722 of housing 718 and an arm 730 extending from flange 728. Rear housing 722, flange 728 and arm 730 together define a slot 732. Other types of mounts 724 may alternatively be utilized for attachment of contextual display device 700 to an asset 126 including, e.g., hook and loop fasteners, a snap-fit arrangement, a magnetic attachment, brackets, suction cups, rubber straps or bands, slide-in rails or tracks or any other attachment mechanism.

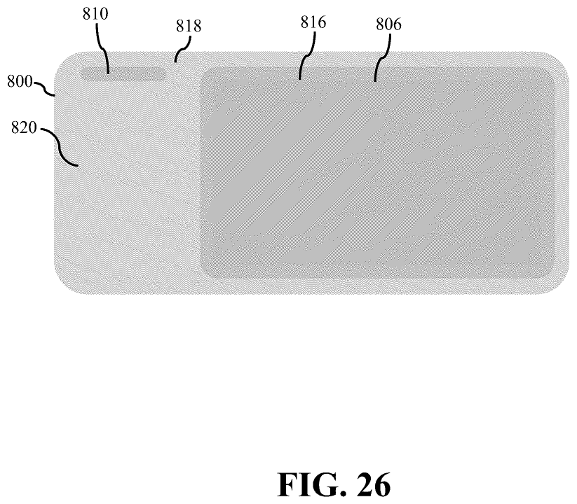

With reference to FIG. 26, a contextual display device 800 is described. Contextual display device 800 comprises similar features to contextual display device 100 where like reference numbers refer to similar components and functionality except as described below.

Contextual display device 800 comprises a housing 818 including the same or similar circuitry 114 (FIG. 1) to that found in contextual display device 100. Contextual display device 800 and housing 818 may also comprise other common components such as, e.g., a charging port, ventilation holes, wireless charging technology or other similar components.

Housing 818 comprises a front housing 820 and a rear housing similar to rear housing 722. Front housing 820 and the rear housing may comprise separate components that are joined together or alternatively may comprise two portions of a single component.

Front housing 820 comprises an optical substrate 816 of a display 706 and a light source 810. The rear housing of housing 818 comprises a mount similar to mount 724 of housing 718 that is configured for attachment to an asset 126 (FIG. 7) in need of contextual information such as, e.g., a surgical case cart as shown. Other mounting technologies and techniques such as those described above for mounts 124, 624 and 724 may also or alternatively be utilized. In this embodiment, no physical input device, such as a button or other actuatable element, is present on front housing 820. In some embodiments, optical substrate 816 or display 806 may comprise touch sensitive technology to function as an input device. In other embodiments, inputs may alternatively only be received wirelessly, e.g., from scan device 200, server 300 or EHR system 400.

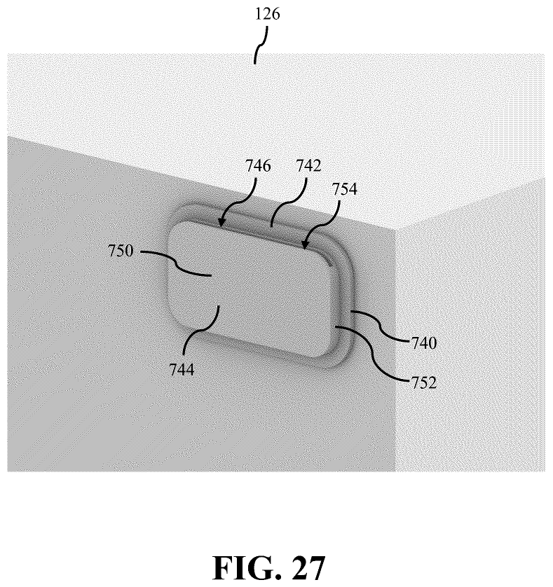

With reference to FIGS. 27-30, an example mount bracket 740 that is configured to receive mount 724 for removable attachment either of contextual display device 700 and contextual display device 800 to asset 126 will be described.

Mount bracket 740 comprises a base plate 742 and a receiving plate 744 extending from or extruded from base plate 742. Receiving plate 742 comprises a slot 746 that is configured for receiving a portion of arm 730 of mount 724. A portion 750 of receiving plate 744 is also configured for positioning within slot 732 of mount 724 when arm 730 is received within slot 746. Receiving plate 742 further comprises side walls 752 that are configured to inhibit lateral movement of arm 730 when arm 730 is received within slot 746. For example, in some embodiments, receiving plate 744 may be configured for insertion of arm 730 in a vertical direction, e.g., through an opening 754 if slot 746, while inhibiting movement and removal of arm 730 from slot 746 in a lateral or side-to-side direction. Mount bracket 740 may be secured to asset 126 using an attachment mechanism 748, for example, an adhesive, hook and loop fasteners, a snap-fit arrangement, a magnetic attachment, brackets, suction cups, slide-in rails or tracks or any other attachment mechanism.

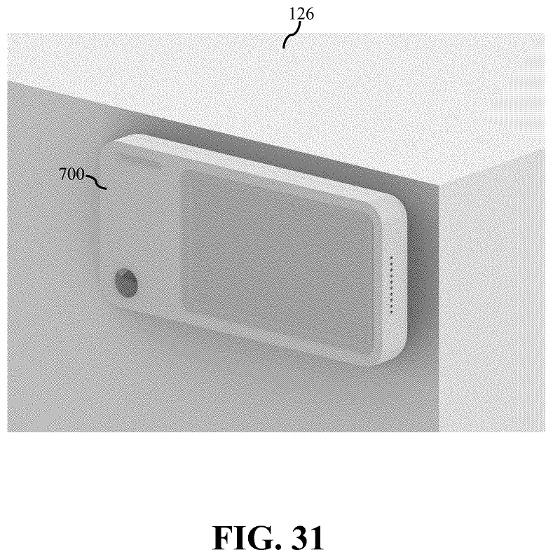

As shown in FIG. 31, housing 718 is secured to mount bracket 740 by the insertion of arm 730 of mount 724 through opening 754 into slot 746 of mount bracket 740, and the insertion of the portion of receiving plate 744 of mount bracket 740 into slot 732 of mount 724. In this manner, contextual display devices 700 and 800 may be removably attached to asset 126 and provide contextual information to a user of asset 126 such as hospital staff or any other user in need of contextual information about asset 126.

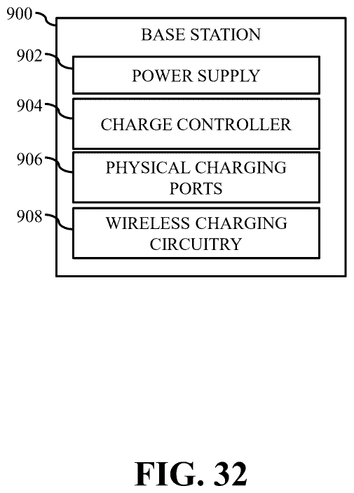

With reference to FIGS. 32-36, an example base station 900 for storing and charging multiple contextual display devices 700 will be described. While described with reference to contextual display device 700, any of contextual display devices 100, 600 and 800 may also or alternatively be stored and charged using base station 900.

Base station 900 comprises a power supply 902, a charge controller 904, physical charging ports 906, wireless charging circuitry 908 and other circuitry commonly found in circuitry for charging electronic devices. In some embodiments, both physical charging ports 906 and wireless charging circuitry 908 may be included in base station 900. In other embodiments, either physical charging ports 906 or wireless charging circuitry 908, but not both, may be included in base station 900.

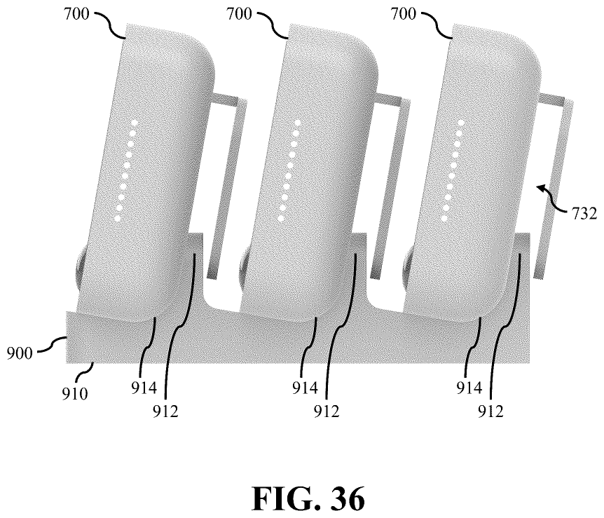

Base station 900 comprises a platform 910 and walls 912 extending from platform 910. Platform 910 and walls 912 together define device seats 914 that are configured to receive contextual display devices 700. In some embodiments, seats 914 may comprise contours or other features that mirror or match to corresponding contours of contextual display devices 700, e.g., curvature or other features that are configured to enable stable seating on seats 914, for example, as shown in FIG. 36. Walls 912 are configured and dimensioned for receipt within slots 732 of contextual display devices 700 and may be utilized to further secure contextual display devices 700 when positioned on base station 900.

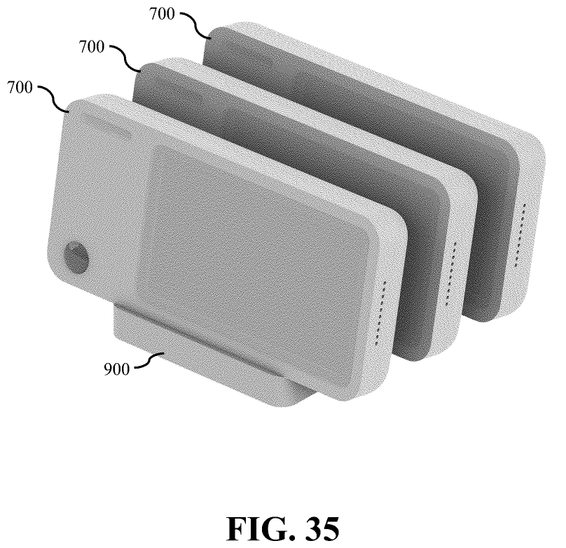

As shown in FIGS. 35 and 36, for example, multiple contextual display devices 700 may be positioned on base station 900 on seats 914 to for an electrical connection with base station 900 and enable charging of batteries of contextual display device 700 via physical charging ports 906 or wireless charging circuitry 908.

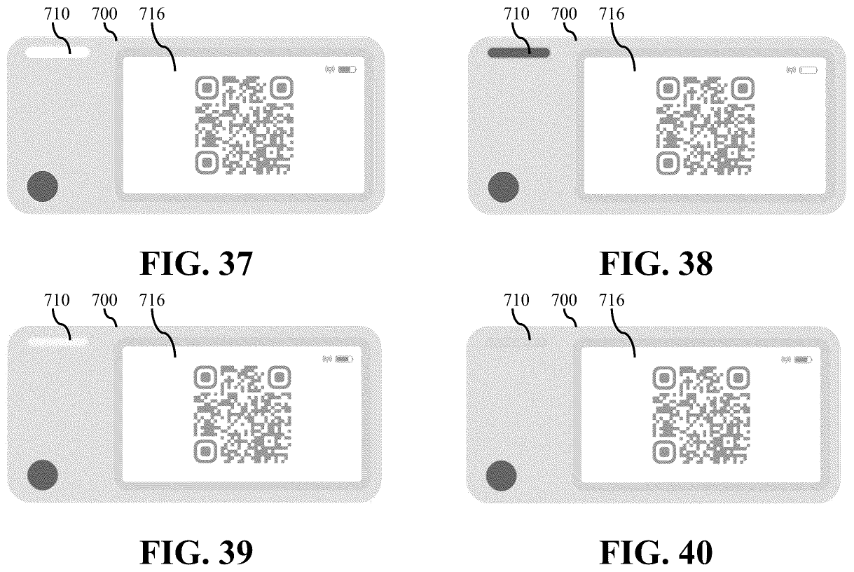

With reference to FIGS. 37-48, the display of contextual information on contextual display device 700 will now be described in the context of a surgical case cart as asset 126. While described with reference to contextual display device 700, the display of contextual information may also or alternatively be performed by any of contextual display devices 100, 600 and 800. While described with reference to a surgical case cart as asset 126, any other contextual information may be presented on contextual display device 700 for other assets 126 in other embodiments.

With reference to FIG. 37, when not in use, or when a user has not been authorized or validated to read contextual information on contextual display device 700 recently by server 300 or EHR system 400, contextual display device 700 may present a machine-readable code such as, e.g., a barcode, QR code or other digital information, on optical substrate 716 that may be scanned by a user. Even when in this state, contextual display device 700 may present device related information such as, e.g., a battery state. In this state, absent any other contextual information to be presented, light source 710 may be turned off.

With reference to FIGS. 38-40, for example, contextual display device 700 is configured to present contextual information regarding a variety of battery states to a user using light source 710, optical substrate 716, or both.

As an example, with reference to FIG. 38, when in a very low battery state, e.g., less than 20% battery or another percentage, light source 710 may turn a particular color, e.g., red, and a representation 717 of battery state on optical substrate 716 may be updated to represent an almost empty battery or another symbol or other information indicative of battery life. In some embodiments, representation 717 may also or alternatively include a percentage of battery life remaining.

With reference to FIG. 39, when in a moderate battery state, e.g., between 20% battery and 70% or another percentage, light source 710 may turn a particular color, e.g., yellow, and representation 717 of battery state on optical substrate 716 may be updated to represent a partially full battery or another symbol or other information indicative of battery life.

With reference to FIG. 40, when in a full or almost full battery state, e.g., between 70% battery and 100% or another percentage, light source 710 may turn a particular color, e.g., green, and representation 717 of battery state on optical substrate 716 may be updated to represent a full or mostly full battery or another symbol or other information indicative of battery life.

In some embodiments, the ranges of percentage for each color of light source 710 may be different or user configurable. In an embodiment, for example, a user may configure red to correspond to between 0% and 20%, yellow to correspond between 20% and 40%, green to correspond to between 40% and 60% and white or turned off light source to corresponding to 60% to 100%. Any other colors for battery states or other contextual information may also or alternatively be utilized and in some embodiments may also be user configurable.

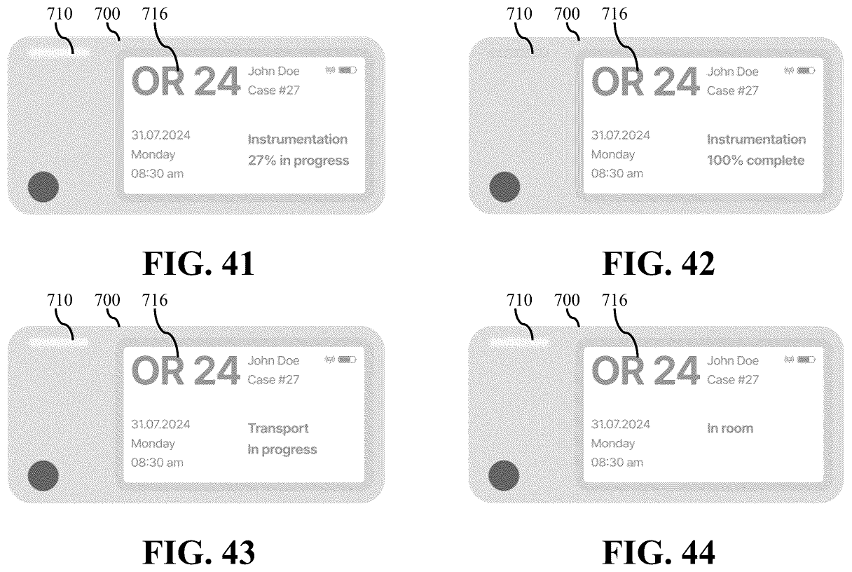

With reference to FIGS. 41-48, example contextual information about asset 126 to which contextual display device 700 is attached is described.

At FIG. 41, for example, contextual display device 700 has been attached to and assigned to an asset 126, such as a surgical case cart. The assignment may be performed by attaching contextual display device 700 to asset 126 and scanning a machine-readable code, e.g., a QR code as shown in FIG. 41, presented on optical substrate 716 using scan device 200. As part of the scan, the user instructs server 300 to correlate contextual display device 700 to asset 126 and the patient's patient data 502 in EHR system 400 in association with a pending surgery.

In some embodiments, a separate machine-readable code found on asset 126 may also be scanned by scan device 200 as part of the assignment. For example, the machine-readable code found on asset 126 may be scanned to identify the asset 126 that contextual display device 700 is being attached to. In some embodiments, the mounting bracket attached to asset 126, e.g., mounting bracket 740 in this example, may comprise the machine-readable code for asset 126 that may be scanned prior to attachment of contextual display device 700 to asset 126.

In some embodiments, contextual display device 700 may be configured to identify asset 126 automatically as part of being attached to asset 126, e.g., using NFC, magnetic technologies, RFID technologies or other similar short range or contact based technologies. As an example, a scan of contextual display device 700, once attached to asset 126, may also provide information on the attached asset 126, e.g., by presenting a modified machine-readable code that includes identifying information about both contextual display device 700 and asset 126. In another example, a scan of contextual display device 700, once attached to asset 126, may cause contextual display device 700 to provide information about the attached asset 126 to server 300 separate from scan device 200.

Once contextual display device 700 is correlated to asset 126 and the patient's patient data 502, server 300 provides contextual information to contextual display device 700 for presentation on optical substrate 716. As an example, server 300 may access the patient's patient data 502 which may include an indication of which surgical instruments or other supplies are required for the patient's surgical operation. Server 300 may obtain this information and store it as patient data 308. Patient data 308 may then be supplemented with an indication of which of those surgical instruments or other supplies have been assigned to the patient and added to asset 126-96+

These surgical instruments may also be scanned by scan device 200 to associate them with patient data 308 and to associate them with asset 126. Server 300 may also update patient data 502 based on updates to patient data 308 in some embodiments. The surgical instruments or other supplies may then physically added to asset 126 by the staff member using scan device 200, e.g., placed in the surgical case cart. Server 300 may then cause contextual display device 700 to present an instrumentation progress level, e.g., 27%, for asset 126 as contextual information based on which instruments have already been added to asset 126. For example, server 300 may receive an update from scan device 200 or EHR system 400 regarding the instrumentation progress level for a corresponding asset 126 and then push an update to the corresponding contextual display device 700 for presentation on optical substrate 716 as contextual information. In some embodiments, when additional instrumentation or other supplies are still needed, e.g., the full list of surgical instruments and supplies have not yet been added, light source 710 may be illuminated in a particular color, e.g., yellow, to indicate that asset 126 is not yet ready for use.

At FIG. 42, server 300 determines that all instrumentation and other supplies that are needed for an operation have been added to asset 126. Server 300 may then push an update to contextual display device 700 for presentation on optical substrate 716 as contextual information to show that instrumentation is complete and asset 126 is ready for transport or use. In an embodiment, light source 710 may be illuminated in a particular color, e.g., green, to indicate that asset 126 is ready for use.

At FIG. 43, contextual display device 700 may determine that asset 126 is in motion, e.g., using BLE, geofencing, or other wireless tracking technologies, and change the image presented on optical substrate 716 to indicate that transport is in progress. Light source 710 may also be changed to yellow or another color in some embodiments. In some embodiments, movement of contextual display device 710 may also or alternatively be detected by another device, e.g., a beacon or other hospital tracking system, which may relay the movement to server 300. In this embodiment, server 300 may alternatively push an update to contextual display device 700 for presentation on optical substrate 716 as contextual information to show that asset 126 is in transport. Server 300 may also store this movement information in patient data 308, provide an update on movement of asset 126 to EHR system 400 for storage in patient data 502, or both.

At FIG. 44, contextual display device 700 may determine that asset 126 is located in the assigned room (or another room), e.g., using BLE, geofencing, or other wireless tracking technologies, and change the image presented on optical substrate 716 to indicate that asset 126 is in room. Light source 710 may also be changed to yellow or another color in some embodiments. In some embodiments, the location of contextual display device 710 may also or alternatively be detected by another device, e.g., a beacon or other hospital tracking system, which may relay the location to server 300. In this embodiment, server 300 may determine that asset 126 is in a particular location, e.g., the assigned OR, and push an update to contextual display device 700 for presentation on optical substrate 716 to show that asset 126 is the room. Server 300 may also store this location information in patient data 308, provide an update on location of asset 126 to EHR system 400 for storage in patient data 502, or both.

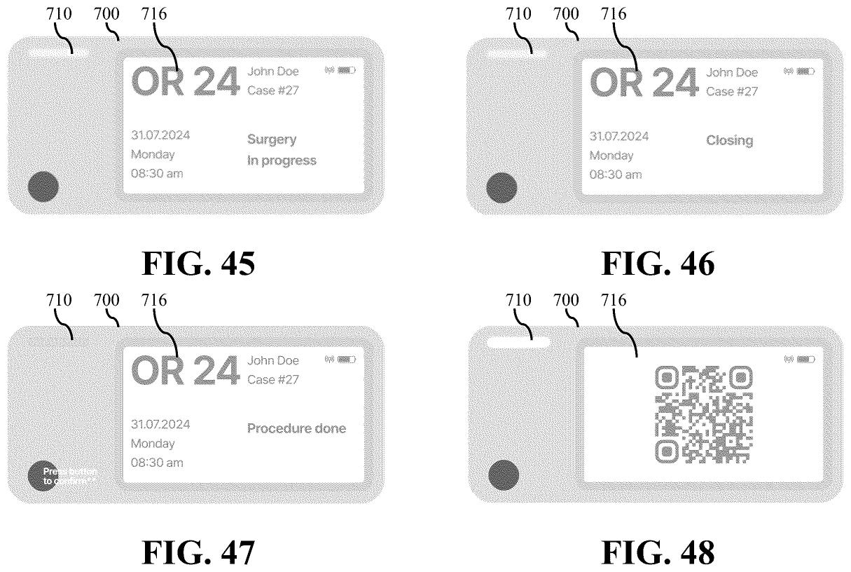

At FIG. 45, server 300 determines that surgery is in progress. As an example, server 300 may obtain an update from EHR system 400 that indicates that staff or a surgeon has initiated surgery for the corresponding patient in the location of asset 126. Server 300 may then push an update to contextual display device 700 for presentation on optical substrate 716 to show that surgery is in progress. In an embodiment, light source 710 may be illuminated in a particular color, e.g., yellow, to indicate that surgery using asset 126 is in progress.

At FIG. 46, server 300 determines that surgery is closing. As an example, server 300 may obtain an update from EHR system 400 that indicates that staff or a surgeon has initiated closing of the surgery for the corresponding patient in the location of asset 126. Server 300 may then push an update to contextual display device 700 for presentation on optical substrate 716 to show that surgery is closing. In an embodiment, light source 710 may be illuminated in a particular color, e.g., yellow, to indicate that surgery using asset 126 is closing.

At FIG. 47, server 300 determines that the surgical procedure is complete. As an example, server 300 may obtain an update from EHR system 400 that indicates that staff or a surgeon has submitted a completion of surgery to EHR system 400 for the corresponding patient in the location of asset 126. Server 300 may then push an update to contextual display device 700 for presentation on optical substrate 716 to show that the procedure is complete. In an embodiment, light source 710 may be illuminated in a particular color, e.g., green, to indicate that the surgical procedure using asset 126 is complete.

At FIG. 48, contextual display device 700 may determine that asset 126 is in motion out of the operating room, e.g., using BLE, geofencing, or other wireless tracking technologies, and change the image presented on optical substrate 716. For example, in some embodiments, now that the surgical procedure is complete, contextual display device 700 may revert to a waiting state and present a machine-readable code, e.g., a QR code or another scannable identifier, to indicate that asset 126 is not usable for another operation. Light source 710 may also be changed to white, off, or another color in some embodiments. In some embodiments, movement of contextual display device 710 may also or alternatively be detected by another device, e.g., a beacon or other hospital tracking system, which may relay the movement to server 300. In this embodiment, server 300 may alternatively push an update to contextual display device 700 for presentation on optical substrate 716 to show that asset 126 is no longer available for use. Server 300 may also store this movement information in patient data 308, provide an update on movement of asset 126 to EHR system 400 for storage in patient data 502, or both.

In some embodiments, movement of asset 126 out of the operating room may alternatively cause light source 710 to turn red or another color and optical substrate 716 to present an indication that asset 126 is in a post-surgical state and in need of cleaning or sanitization. For example, server 300 may unassign asset 126 from the patient and instead assign asset 126 to a cleaning or sanitization program, location, or unit within the hospital. Corresponding contextual information may then be pushed by server 300 to contextual display device 700 for presentation on optical substrate 716 so that a user such as a staff member seeing the contextual information presented on optical substrate 716 can easily determine where to take asset 126 next.

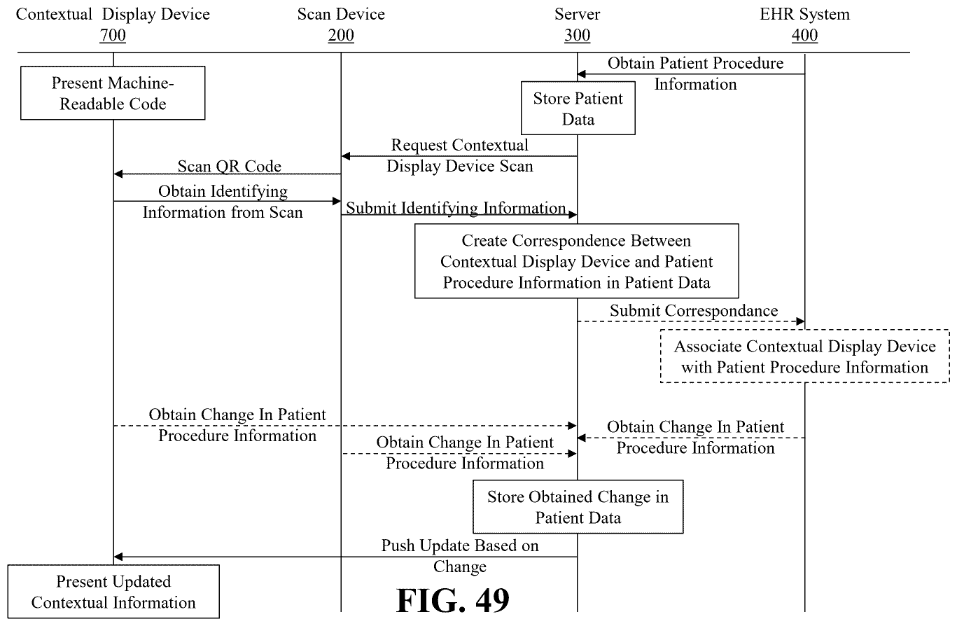

With reference to FIG. 49, an example signal diagram showing interactions between contextual display device 700, scan device 200, server 300 and EHR system 400 will now be described. In other embodiments, any of contextual display devices, 100, 600 and 800 may alternatively be utilized instead of contextual display device 700.

In an initial state, contextual display device 700 presents a machine-readable code such as a QR code or another data image. Server 300 obtains patient procedure information from patient data 502 that indicates that the patient has an upcoming scheduled procedure, e.g., from patient data store 500 via EHR server 400 and stores the patient procedure information as patient data 308. In some embodiments, the patient procedure information may comprise, e.g., a list of instrumentation and other supplies needed for the surgical procedure, an operating room assigned to the procedure, other information about the surgical procedure such as a date, a time, or other similar information, patient information such as a case number, name, allergies, notes or any other information.

Server 300 submits a request to scan device 200 to scan contextual display device 700. In some embodiments, server 300 may also or alternatively submit a notification to a nurse or other staff member indicating that the nurse should use a scan device 200 to scan contextual display device 700. For example, the notification may be on scan device 200, via email, via a chat application, or in any other manner operated by the hospital.

The nurse or other staff member activates scan device 200 to scan the machine-readable code presented on contextual display device 700 and obtains identifying information about contextual display device 700 from the scan, e.g., a unique identifier or other identifying information.

In some embodiments, identifying information for asset 126 may also be obtained, e.g., by scanning a corresponding machine-readable code, or in another manner such as described above.

Scan device 200 submits the identifying information to server 300 and server 300 creates a correspondence between contextual display device 700, asset 126 and the patient procedure information in patient data 308. As an example, the identifying information of contextual display device 700 may be add into the patient procedure information stored in patient data 308. In some embodiments, server 300 may also submit the created correspondence to EHR system 400 for association and addition to patient data 502.

Server 300 obtains a change in the patient procedure information, e.g., based on the addition of one or more surgical tools or supplies to asset 126 to which contextual display device 700 is attached. In some embodiments, when a surgical instrument or other supply is scanned, e.g., by scan device 200, scan device 200 may also be utilized to scan contextual display device 700, or asset 126, to create an association of that surgical instrument or supply with contextual display device 700 in patient data 308. In other embodiments, the act of scanning and adding surgical instruments to the patient procedure information or medical record may automatically associate that surgical instrument as having been added to asset 126 that corresponds to contextual display device 700 in patient data 308. Server 300 may also be configured to provide an update to EHR system 400 based on any changes in patient data 308 for inclusion in patient data 502.

Other examples of a change include a determination that contextual display device 700 is in transit, in a particular location, a scan or entry by a doctor, nurse or staff member on another device related to the patient procedure information, e.g., begin surgery, surgery in process, surgery closing, surgery complete, or another update. Server 300 may obtain such changes from contextual display device 100, scan device 200, EHR system 400, from other devices within a hospital or other setting, or in any other manner.

As an example, server 300 obtains one or more changes in the patient procedure information from contextual display device 100, scan device 200, EHR system 400 or another source, stores the change in patient data 308, and pushes an update to contextual display device 700 based on the change. Contextual display device 700 may then present updated contextual information on optical substrate 716 based on the update, e.g., in the manner shown in FIGS. 41-48.

The particular processing operations and other system functionality described in conjunction with the FIG. 49 are presented by way of illustrative example only and should not be construed as limiting the scope of the disclosure in any way. Alternative embodiments can use other types of processing operations. For example, the ordering of the process steps may be varied in other embodiments, or certain steps may be performed at least in part concurrently with one another rather than serially. Also, one or more of the process steps may be repeated periodically, or multiple instances of the process can be performed in parallel with one another in order to implement the disclosed embodiments.

Functionality such as that described in conjunction with the process of FIG. 49 may be implemented at least in part in the form of one or more software programs stored in memory and executed by a processor of a processing device such as a computer or server. As will be described herein, a memory or other storage device having executable program code of one or more software programs embodied therein is an example of what is more generally referred to herein as a “processor-readable storage medium.”

FIGS. 1 through 49 are conceptual illustrations allowing for an explanation of the disclosed embodiments of the invention. Notably, the figures and examples above are not meant to limit the scope of the invention to a single embodiment, as other embodiments are possible by way of interchange of some or all of the described or illustrated elements. Moreover, where certain elements of the disclosed embodiments can be partially or fully implemented using known components, only those portions of such known components that are necessary for an understanding of the disclosed embodiments are described, and detailed descriptions of other portions of such known components are omitted so as not to obscure the disclosed embodiments. In the present specification, an embodiment showing a singular component should not necessarily be limited to other embodiments including a plurality of the same component, and vice-versa, unless explicitly stated otherwise herein. Moreover, terms in the specification or claims are not intended to be ascribed an uncommon or special meaning unless explicitly set forth as such. Further, the disclosed embodiments encompass present and future known equivalents to the known components referred to herein by way of illustration.

It should be understood that the various aspects of the embodiments could be implemented in hardware, firmware, software, or combinations thereof. In such embodiments, the various components and/or steps would be implemented in hardware, firmware, and/or software to perform the functions of the disclosed embodiments. That is, the same piece or different pieces of hardware, firmware, or module of software could perform one or more of the illustrated blocks (e.g., components or steps). In software implementations, computer software (e.g., programs or other instructions) and/or data is stored on a machine-readable medium as part of a computer program product and is loaded into a computer system or other device or machine via a removable storage drive, hard drive, or communications interface. Computer programs (also called computer control logic or computer-readable program code) are stored in a main and/or secondary memory, and executed by one or more processors (controllers, or the like) to cause the one or more processors to perform the functions of the invention as described herein. In this document, the terms “machine readable medium,” “computer-readable medium,” “computer program medium,” and “computer usable medium” are used to generally refer to media such as a random access memory (RAM); a read only memory (ROM); a removable storage unit (e.g., a magnetic or optical disc, flash memory device, or the like); a hard disk; or the like.

The foregoing description will so fully reveal the general nature of the disclosed embodiments that others can, by applying knowledge within the skill of the relevant art(s) (including the contents of the documents cited and incorporated by reference herein), readily modify and/or adapt for various applications such specific embodiments, without undue experimentation, without departing from the general concept of the disclosed embodiments. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

What is claimed is:

1. A contextual display device comprising:
a housing comprising:
a mount extending from the housing that is configured for removable attachment to a mount bracket of an asset of a medical facility;
a communication interface that is configured to communicate with a server, the server being configured to access patient data stored in an electronic health record system;
a display, the display being configured to present contextual information;
at least one processor in communication with the communication interface and the display, the at least one processor being configured to:
cause the display to present a machine-readable code;
obtain contextual information corresponding to the patient data from the server via the communication interface, the contextual display device being associated with the patient data by the server based on a scan of the machine-readable code by a scan device; and
cause the display to present the contextual information.

2. The contextual display device of claim 1, wherein the at least one processor is further configured to:
obtain updated contextual information corresponding to the patient data from the server via the communication interface; and
cause the display to present the updated contextual information.

3. The contextual display device of claim 1 wherein the contextual information corresponding to the patient data comprises contextual information about a status of the asset.

4. The contextual display device of claim 3 wherein the contextual information corresponding to the patient data comprises an instrumentation readiness status corresponding to a list of surgical instruments to be added to the asset.

5. The contextual display device of claim 3 wherein the contextual information corresponding to the patient data comprises location information corresponding to a location of the asset.

6. The contextual display device of claim 3 wherein the contextual information corresponding to the patient data comprises activity information corresponding to a use of the asset.

7. The contextual display device of claim 6 wherein the activity information comprises an operating room status corresponding to the use of the asset.

8. A server comprising:
memory;
a communication interface that is configured to interface with an electronic health record system, a contextual display device removably attached to an asset, and a scan device;
at least one processor that is configured to:
access patient data of a patient in the electronic health record system;
obtain identifying information of the contextual display device from the scan device, the identifying information being generated based on a scan of a machine-readable code presented on a display of the contextual display device by the scan device;
associate the contextual display device with the patient data based on the identifying information;
obtain contextual information corresponding to the patient data from the electronic health record system via the communication interface; and
transmit a command including the contextual information to the contextual display device, the command being configured to instruct the contextual display device to present the contextual information on the display.

9. The server of claim 8, wherein the at least one processor is further configured to:
obtain updated contextual information corresponding to the patient data from the electronic health record system via the communication interface; and
transmit a second command including the updated contextual information to the contextual display device, the second command being configured to instruct the contextual display device to present the updated contextual information on the display.

10. The server of claim 8 wherein the contextual information corresponding to the patient data comprises contextual information about a status of the asset.

11. The server of claim 10 wherein the contextual information corresponding to the patient data comprises an instrumentation readiness status corresponding to a list of surgical instruments to be added to the asset.

12. The server of claim 10 wherein the at least one processor is configured to:
obtain a location information corresponding to a location of the contextual display device via the communication interface;
provide an update to the patient data corresponding to the location information; and
transmit a second command including updated contextual information corresponding the location information to the contextual display device the second command being configured to instruct the contextual display device to present the updated contextual information on the display including an indication of the location of the contextual display device.

13. The server of claim 10 wherein the contextual information corresponding to the patient data comprises activity information corresponding to a use of the asset.

14. The server of claim 13 wherein the activity information comprises an operating room status corresponding to the use of the asset.

15. The server of claim 8 wherein the obtaining the identifying information of the contextual display device from the scan device comprises providing an instruction to the scan device to scan the machine-readable code presented on the display of the contextual display device, the scan device being configured to present the instruction to a user of the scan device.

16. A non-transitory computer-readable medium storing instructions that, when executed by at least one processor, cause the at least one processor to:
access patient data of a patient in an electronic health record system;
obtain identifying information of a contextual display device removably attached to an asset from a scan device, the identifying information being generated based on a scan of a machine-readable code presented on a display of the contextual display device by the scan device;
associate the contextual display device with the patient data based on the identifying information;
obtain contextual information corresponding to the patient data from the electronic health record system; and
transmit a command including the contextual information to the contextual display device, the command being configured to instruct the contextual display device to present the contextual information on the display.

17. The non-transitory computer-readable medium of claim 16, wherein the instructions, when executed by the at least one processor, cause the at least one processor to:
obtain updated contextual information corresponding to the patient data from the electronic health record system; and
transmit a second command including the updated contextual information to the contextual display device, the second command being configured to instruct the contextual display device to present the updated contextual information on the display.

18. The non-transitory computer-readable medium of claim 16 wherein the contextual information corresponding to the patient data comprises contextual information about a status of the asset.

19. The non-transitory computer-readable medium of claim 18 wherein the contextual information about the status of the asset comprises at least one of:
an instrumentation readiness status corresponding to a list of surgical instruments to be added to the asset; and
location information corresponding to a location of the asset; and
activity information corresponding to a use of the asset.

20. The non-transitory computer-readable medium of claim 16 wherein obtaining the identifying information of the contextual display device from the scan device comprises providing an instruction to the scan device to scan the machine-readable code presented on the display of the contextual display device, the scan device being configured to present the instruction to a user of the scan device.
